(19) United States
(12) Patent Application Publication
(10) Pub. No.: US 2026/0157684 A1
(43) Pub. Date: Jun. 11, 2026

(54) MULTI-LEVEL MECHANORECEPTOR DEVICE-BASED STIFFNESS MEASUREMENT SENSOR AND METHOD OF DRIVING THE SENSOR

(71) Applicant: KOREA INSTITUTE OF SCIENCE AND TECHNOLOGY, Seoul (KR)

(72) Inventors: Hyun Jung YI, Seoul (KR); Su Youn LEE, Seoul (KR); Min Seok OH, Seoul (KR); Ju Hee KIM, Seoul (KR); Jin Young JEONG, Seoul (KR); Sang Heon KIM, Seoul (KR)

(73) Assignee: KOREA INSTITUTE OF SCIENCE AND TECHNOLOGY, Seoul (KR)

(21) Appl. No.: 18/710,144

(22) PCT Filed: Jan. 19, 2024
(86) PCT No.: PCT/KR2024/000930

§ 371 Date: May 14, 2024

(30) Foreign Application Priority Data
Jan. 10, 2024 (KR) 10-2024-0004179

(51) Int. Cl. A61B 5/00

(52) U.S. Cl. CPC A61B 5/442

(57) ABSTRACT

A multi-level mechanoreceptor device-based stiffness measurement sensor according to an embodiment includes a substrate, a contact portion disposed below the substrate and partially spaced apart from the substrate, and an electrode portion disposed at a lower end of the substrate and disposed corresponding to the contact portion, in which an area where the electrode portion and the contact portion are in contact gradually increases as an external force is applied to an object to be measured at a bottom part of the contact portion.

### TECHNICAL FIELD

The following embodiments relate to a multi-level mechanoreceptor device-based stiffness measurement sensor and a method of driving the same.

### BACKGROUND ART

In the case of commercial products to measure the stiffness of soft tissue such as skin, there is a disadvantage that an error is large by measuring a characteristic of skin in a static situation using a method of measuring a force applied when a product presses the skin to a certain height.

In addition, there is technology that measures the stiffness in a dynamic situation based on a characteristic that a speed at which a force is transmitted is proportional to the stiffness when a force is applied at a constant speed, but the technology has a disadvantage of being difficult to miniaturize because the force application speed must be uniformly applied at a predetermined value.

Accordingly, there is a need for technology that may measure the stiffness of a material regardless of the force application speed.

Korean Patent Application Publication No. 10-2022-0131595 discloses a device and method for measuring skin elasticity.

### DISCLOSURE OF THE INVENTION

### Technical Goals

The purpose according to an embodiment is to provide a multi-level mechanoreceptor device-based stiffness measurement sensor that may derive the stiffness of an object to be measured without being affected by a speed at which an external force is applied to the object to be measured.

The purpose according to an embodiment is to provide a multi-level mechanoreceptor device-based stiffness measurement sensor that is manufactured in a small size and has no problem with durability of the device due to detachment and/or attachment.

### Technical Solutions

A multi-level mechanoreceptor device-based stiffness measurement sensor according to an embodiment includes a substrate, a contact portion disposed below the substrate and partially spaced apart from the substrate, and an electrode portion disposed at a lower end of the substrate and disposed corresponding to the contact portion, in which an area where the electrode portion and the contact portion are in contact gradually increases as an external force is applied to an object to be measured at a bottom part of the contact portion.

The contact portion may include a first elastic member and a second elastic member capable of being pressed by the external force, in which the first elastic member may be thicker than the second elastic member, in which a lower end of the first elastic member may contact an upper surface of the object to be measured, and in which a lower end of the second elastic member may be spaced apart from the upper surface of the object to be measured.

The first elastic member may include a 1-1 protruding element positioned at an upper end of the first elastic member, the second elastic member may include a 2-1 protruding element and a 2-2 protruding element positioned at an upper end of the second elastic member, and the 2-1 protruding element may have a protruding length that is longer than that of the 2-2 protruding element, the electrode portion may include a first electrode member, a second electrode member, and a third electrode member disposed at a position corresponding to the 1-1 protruding element, the 2-1 protruding element, and the 2-2 protruding element, respectively.

When the external force is applied to the object to be measured for a first time, the object to be measured may contract and the first elastic member may contract so that the 1-1 protruding element may begin to contract at a first time point, and thus a contact resistance of the 1-1 protruding element with the first electrode member may rapidly decrease, when the external force is applied to the object to be measured for a second time, the object to be measured may contract, and the first elastic member and the second elastic member may contract so that the 2-1 protruding element may begin to contract at a second time point, and thus a contact resistance of the 2-1 protruding element with the second electrode member may rapidly decrease, the external force is applied to the object to be measured for a third time, the object to be measured may contract, and the first elastic member and the second elastic member may contract so that the 2-2 protruding element may begin to contract at a third time point, and thus a contact resistance of the 2-2 protruding element with the third electrode member may rapidly decrease, in which the second time may be defined as a time from the first time point to the second time point, and the third time may be defined as a time from the second time point to the third time point.

The second time may be inversely proportional to a speed at which the external force is applied to the contact portion, and the third time may be inversely proportional to a speed at which the external force is applied to the contact portion and stiffness of the object to be measured.

A ratio of the second time to the third time may be proportional to the stiffness of the object to be measured.

The multi-level mechanoreceptor device-based stiffness measurement sensor may further include a first switching element, a second switching element, and a third switching element connected to the first electrode member, the second electrode member, and the third electrode member, respectively, in which each of the first switching element, the second switching element, and the third switching element may switch on when a size of a contact resistance decreases to be less than or equal to a predetermined level.

Each of the 1-1 protruding element, the 2-1 protruding element, and the 2-2 protruding element may include a plurality of protruding elements, and each of the first electrode member, the second electrode member, and the third electrode member may form a closed circuit.

A method of driving a multi-level mechanoreceptor device-based stiffness measurement sensor according to an embodiment includes applying an external force to an object to be measured for a first time so that a 1-1 protruding element begins to be compressed at a first time point, and thus a contact resistance of the 1-1 protruding element with a first electrode member rapidly decreases, applying the external force to the object to be measured for a second time so that a 2-1 protruding element begins to be compressed at a second time point, and thus a contact resistance of the 2-1 protruding element with a second electrode member rapidly decreases, applying the external force to the object to be measured for a third time so that a 2-2 protruding element begins to be compressed at a third time point, and thus a contact resistance of the 2-2 protruding element with a third electrode member rapidly decreases, and deriving stiffness of the object to be measured from a ratio of the second time to the third time.

### Effects

A multi-level mechanoreceptor device-based stiffness measurement sensor according to an embodiment may derive the stiffness of an object to be measured without being affected by a speed at which an external force is applied to the object to be measured.

A multi-level mechanoreceptor device-based stiffness measurement sensor according to an embodiment may be manufactured in a small size and there may be no problem with durability of the device due to detachment and/or attachment.

### BRIEF DESCRIPTION OF DRAWINGS

FIG. 1 illustrates a structure of a multi-level mechanoreceptor device-based stiffness measurement sensor, according to an embodiment.

FIG. 2 illustrates a view at a first time point when a 1-1 protruding element begins to be compressed, according to an embodiment.

FIG. 3 illustrates a view at a second time point when a 2-1 protruding element begins to be compressed, according to an embodiment.

FIG. 4 illustrates a view at a third time point when a 2-2 protruding element begins to be compressed, according to an embodiment.

FIG. 5 is a graph illustrating voltage over time, according to an embodiment.

FIG. 6 schematically illustrates a circuit diagram connecting a first electrode member to a first switching element, according to an embodiment.

FIG. 7 is a cross-sectional view taken along line A-A of FIG. 1 to show a circuit diagram in which first to fourth electrode members are electrically connected to each other, according to an embodiment.

FIG. 8 is a flowchart illustrating a method of driving a multi-level mechanoreceptor device-based stiffness measurement sensor, according to an embodiment.

### BEST MODE FOR CARRYING OUT THE INVENTION

Hereinafter, embodiments will be described in detail with reference to the accompanying drawings. However, various alterations and modifications may be made to the embodiments. Here, the embodiments are not construed as limited to the disclosure. The embodiments should be understood to include all changes, equivalents, and replacements within the idea and the technical scope of the disclosure.

The terminology used herein is for the purpose of describing particular embodiments only and is not to be limiting of the embodiments. The singular forms “a,” “an,” and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms including technical and scientific terms used herein have the same meaning as commonly understood by one of ordinary skill in the art to which the embodiments belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

When describing the embodiments with reference to the accompanying drawings, like reference numerals refer to like components and a repeated description related thereto will be omitted. In the description of embodiments, detailed description of well-known related structures or functions will be omitted when it is deemed that such description will cause ambiguous interpretation of the present disclosure.

In addition, terms such as first, second, A, B, (a), (b), and the like may be used to describe components of the embodiments. These terms are used only for the purpose of discriminating one constituent element from another constituent element, and the nature, the sequences, or the orders of the constituent elements are not limited by the terms. When one component is described as being “connected”, “coupled”, or “attached” to another component, it should be understood that one component may be connected or attached directly to another component, and an intervening component may also be “connected”, “coupled”, or “attached” to the components.

The same name may be used to describe an element included in the embodiments described above and an element having a common function. Unless otherwise mentioned, the descriptions on the embodiments may be applicable to the following embodiments and thus, duplicated descriptions will be omitted for conciseness.

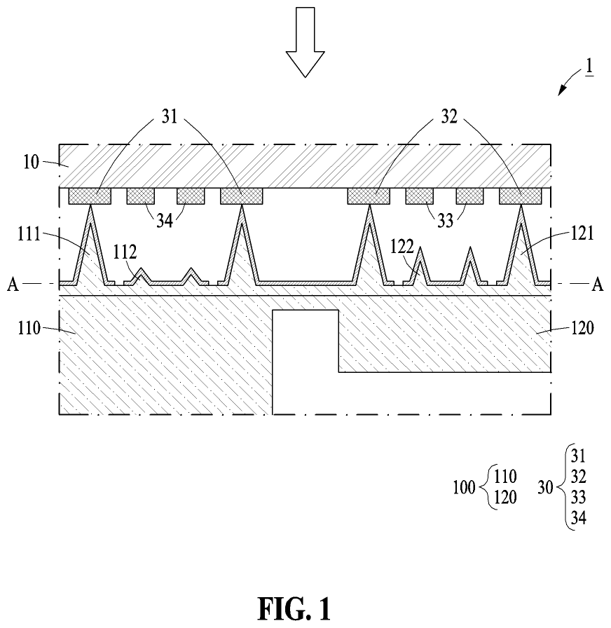

FIG. 1 illustrates a structure of a stiffness measurement sensor 1 based on a multi-level mechanoreceptor device, according to an embodiment.

Referring to FIG. 1, a multi-level mechanoreceptor device-based stiffness measurement sensor 1 (hereinafter, referred to as a “stiffness measurement sensor”) according to an embodiment may include a substrate 10, a contact portion 100 disposed below the substrate 10 and partially spaced apart from the substrate 10, and an electrode portion 30 disposed at a lower end of the substrate 10 and disposed corresponding to the contact portion 100, and an area where the electrode portion 30 and the contact portion 100 are in contact may gradually increase as an external force is applied to an object 20 to be measured at a bottom part of the contact portion 100.

The substrate 10 may be connected to the electrode portion 30 and may form an overall circuit including the electrode portion 30. The substrate 10 may be made of a printed circuit board (PCB) integrated circuit, silicon dioxide (SiO₂), or silicon (Si).

The object 20 to be measured may correspond to soft tissue such as skin.

The contact portion 100 may include a first elastic member 110 and a second elastic member 120 capable of being pressed by the external force, the first elastic member 110 may be thicker than the second elastic member 120, a lower end of the first elastic member 110 may contact an upper surface of the object 20 to be measured, and a lower end of the second elastic member 120 may be spaced apart from the upper surface of the object 20 to be measured. The first elastic member 110 and the second elastic member 120 may be made of a silicon elastic body such as polydimethylsiloxane (PDMS).

The first elastic member 110 may include a 1-1 protruding element 111 and a 1-2 protruding element 112 positioned at an upper end of the first elastic member 110, and the 1-1 protruding element 111 may have a protruding length that is longer than that of the 1-2 protruding element 112. The 1-1 protruding element 111 and the 1-2 protruding element 112 may be coated with a conductive material. Moreover, the conductive material may not be coated between the 1-1 protruding element 111 and the 1-2 protruding element 112 for insulation.

The second elastic member 120 may include a 2-1 protruding element 121 and a 2-2 protruding element 122 positioned at an upper end of the second elastic member 120, and the 2-1 protruding element 121 may have a protruding length that is longer than that of the 2-2 protruding element 122. The 2-1 protruding element 121 and the 2-2 protruding element 122 may be coated with a conductive material. Moreover, the conductive material may not be coated between the 2-1 protruding element 121 and the 2-2 protruding element 122 for insulation.

As described above, the conductive material may be coated at the upper end of the 1-1 protruding element 111, the 1-2 protruding element 112, the 2-1 protruding element 121, and the 2-2 protruding element 122, and the conductive material may contact the electrode portion 30. The 1-1 protruding element 111, the 1-2 protruding element 112, the 2-1 protruding element 121, and the 2-2 protruding element 122 are shown as a triangular pyramid shape in FIG. 1, but this is only an example, and the 1-1 protruding element 111, the 1-2 protruding element 112, the 2-1 protruding element 121, and the 2-2 protruding element 122 may have various shapes such as a hemispherical shape or the like. Here, the conductive material may be formed as single-walled carbon nanotube (SWNT).

Moreover, the 1-1 protruding element 111 and the 2-1 protruding element 121 may have the same length, and it may be desirable that the 2-2 protruding element 122 has a length that is longer than that of the 1-2 protruding element 112.

The electrode portion 30 may include first to fourth electrode members 31 to 34 disposed at a position corresponding to the 1-1 protruding element 111, the 2-1 protruding element 121, the 2-2 protruding element 122, and the 1-2 protruding element 112, respectively. As the external force is applied to the contact portion 100, each of the 1-1 protruding element 111, the 2-1 protruding element 121, the 2-2 protruding element 122, and the 1-2 protruding element 112 may be compressed so that a contact area between the first to fourth electrode members 31 to 34 and the 1-1 protruding element 111, the 2-1 protruding element 121, the 2-2 protruding element 122, and the 1-2 protruding element 112 may gradually increase, and thus a contact resistance of the 1-1 protruding element 111, the 2-1 protruding element 121, the 2-2 protruding element 122, and the 1-2 protruding element 112 with the first to fourth electrode members 31 to 34 may decrease. Moreover, the electrode portion 30 may be made of gold (Au) or the like.

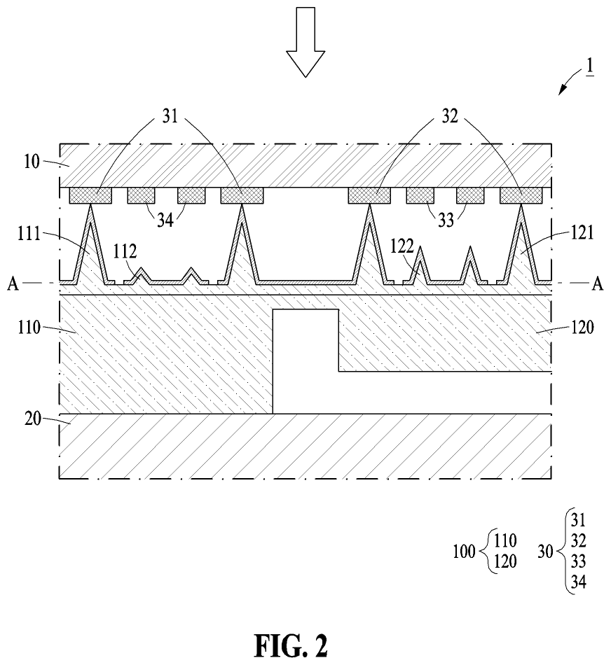

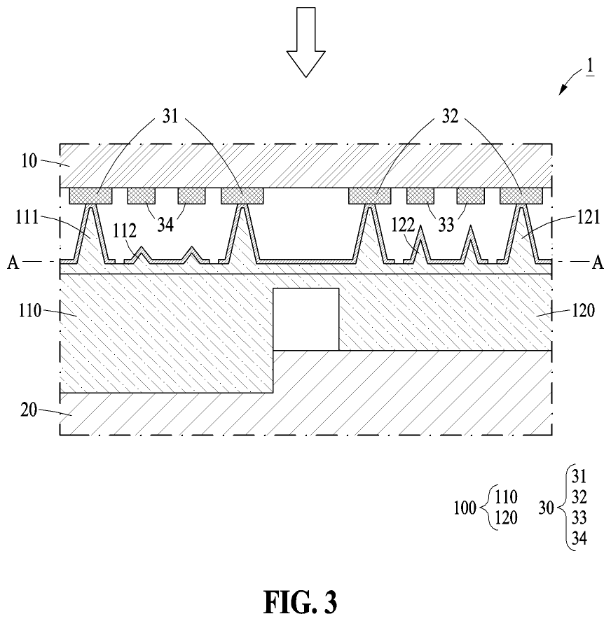

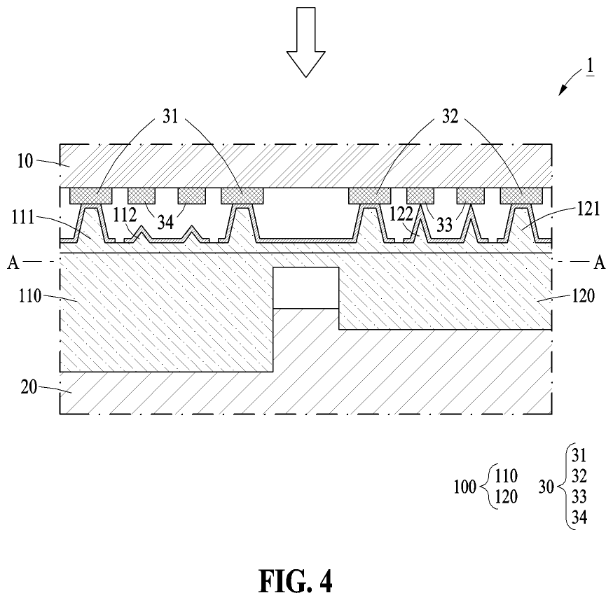

FIG. 2 illustrates a view at a first time point t₁ when the 1-1 protruding element 111 begins to be compressed, according to an embodiment. FIG. 3 illustrates a view at a second time point t₂ when the 2-1 protruding element 121 begins to be compressed, according to an embodiment. FIG. 4 illustrates a view at a third time point t₃ when the 2-2 protruding element 122 begins to be compressed, according to an embodiment.

FIGS. 2 to 4 may correspond to examples of driving the stiffness measurement sensor 1, according to an embodiment.

Referring to FIG. 2, when an external force is applied to the object 20 to be measured for a first time, the object 20 to be measured may contract and the first elastic member 110 may contract so that the 1-1 protruding element 111 may begin to contract at the first time point t₁, and thus a contact resistance of the 1-1 protruding element 111 with the first electrode member 31 may rapidly decrease.

Referring to FIG. 3, when an external force is applied to the object 20 to be measured for a second time t₁₂, the object 20 to be measured may contract, and the first elastic member 110 and the second elastic member 120 may contract so that the 2-1 protruding element 121 may begin to contract at the second time point t₂, and thus a contact resistance of the 2-1 protruding element 121 with the second electrode member 32 may rapidly decrease. Here, as the first elastic member 110 is pressed, the 1-1 protruding element 111 may also be pressed, and thus a contact area between the 1-1 protruding element 111 and the first electrode member 31 may increase.

Referring to FIG. 4, when an external force is applied to the object 20 to be measured for a third time t₂₃, the object 20 to be measured may contract, and the first elastic member 110 and the second elastic member 120 may contract so that the 2-2 protruding element 122 may begin to contract at the third time point t₃, and thus a contact resistance of the 2-2 protruding element 122 with the third electrode member 33 may rapidly decrease. Here, as the first elastic member 110 and the second elastic member 120 are pressed, the 1-1 protruding element 111 and the 2-1 protruding element 121 may also be pressed, and thus a contact area between the 1-1 protruding element 111 and the first electrode member 31 may further increase, and a contact area between the 2-1 protruding element 121 and the second electrode member 32 may increase.

Moreover, here, the second time t₁₂ may be defined as a time from the first time point t₁ to the second time point t₂, and the third time t₂₃ may be defined as a time from the second time point t₂ to the third time point t₃.

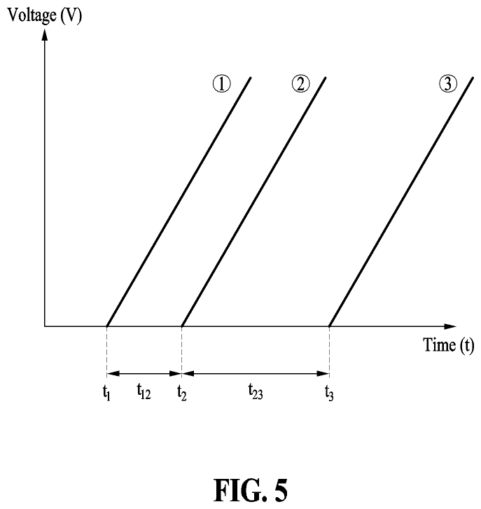

FIG. 5 is a graph illustrating voltage V over time t, according to an embodiment.

The voltage V shown in FIG. 5 may be inversely proportional to contact resistance. That is, for example, in a circuit in which the stiffness measurement sensor 1 and a load resistor are connected in series, the voltage V may correspond to voltage that applies predetermined voltage from outside the circuit to the circuit and is obtained between the load resistors.

Referring to FIG. 5, the second time t₁₂ may correspond to a time from the first time point t₁ when the contact resistance of the 1-1 protruding element 111 with the first electrode member 31 rapidly decreases to the second time point t₂ when the contact resistance of the 2-1 protruding element 121 with the second electrode member 32 rapidly decreases. In addition, the third time t₂₃ may be defined as a time from the second time point t₂ to the third time point t₃ when the contact resistance of the 2-2 protruding element 122 with the third electrode member 33 rapidly decreases.

Moreover, according to an embodiment, the second time t₁₂ may be inversely proportional to a speed v at which an external force is applied to the contact portion 100, and the third time t₂₃ may be inversely proportional to the speed v at which the external force is applied to the contact portion 100, and stiffness k of the object 20 to be measured.

Accordingly, a ratio of the second time t₁₂ to the third time t₂₃ may be proportional to the stiffness k of the object 20 to be measured.

Specifically, as described above, the stiffness k of the object 20 to be measured from the ratio of the second time t₁₂ to the third time t₂₃ may be derived according to Equations 1 to 4 below.

The second time t₁₂ may be expressed according to Equation 1 below.

$${t}_{12} = \frac{{d}_{1}}{v} \qquad [ Equation 1 ]$$

In Equation 1, t₁₂ denotes the second time, v denotes the speed at which the external force is applied to the object 20 to be measured, and d₁ denotes a thickness difference between the first elastic member 110 and the second elastic member 120.

Assuming that the object 20 to be measured and the contact portion 100 are linearly elastic, the third time t₂₃ may be expressed according to Equation 2 below.

$${t}_{23} = \frac{1}{k v} {F}_{p} \qquad [ Equation 2 ]$$

In Equation 2, t₂₃ denotes the third time, k denotes the stiffness of the object 20 to be measured, v denotes the speed at which the external force is applied to the object 20 to be measured, and Fₚ denotes a size of an external force required to press the 2-1 protruding element 121 by a length difference between the 2-1 protruding element 121 and the 2-2 protruding element 122.

Equation 2 may be derived from the fact that a size F of the external force applied to the object 20 to be measured is the same as the size of the external force required to press the 2-1 protruding element 121 by the length difference between the 2-1 protruding element 121 and the 2-2 protruding element 122.

Here, the size F of the external force applied to the object 20 to be measured may be expressed according to Equation 3 below.

$$F = k v {t}_{23} \qquad [ Equation 3 ]$$

In Equation 3, F may correspond to the size of the external force applied to the object 20 to be measured, v may correspond to the speed at which the external force is applied to the object 20 to be measured, and t₂₃ may correspond to the third time.

Accordingly, from Equation 1 and Equation 2, it may be derived that the stiffness k of the object 20 to be measured is proportional to the ratio of the second time t₁₂ to the third time t₂₃, according to Equation 4 below.

$$k = \frac{{t}_{12}}{{t}_{23}} \frac{{F}_{p}}{{d}_{1}} \qquad [ Equation 4 ]$$

In Equation 4, k denotes the stiffness of the object 20 to be measured, t₁₂ denotes the second time, t₂₃ denotes the third time, Fₚ denotes the size of the external force required to press the 2-1 protruding element 121 by the length difference between the 2-1 protruding element 121 and the 2-2 protruding element 122, and d₁ denotes the thickness difference between the first elastic member 110 and the second elastic member 120.

That is, it may be seen that the stiffness k of the object 20 to be measured is proportional to the ratio of the second time t₁₂ to the third time t₂₃, and here, in Equation 5 below, a value of the stiffness k of the object 20 to be measured may be derived by substituting the size Fₚ of the external force to which a general constant C is introduced into Equation 4. That is, the value of the stiffness k of the object 20 to be measured may be derived by introducing a certain constant.

Moreover, as shown in Equation 4, a variable of the speed v at which the external force is applied to the object 20 to be measured may be removed from the ratio of the second time t₁₂ to the third time t₂₃ so that the stiffness measurement sensor 1 may measure the stiffness k of the object 20 to be measured without being affected by the speed v at which the external force is applied to the object 20 to be measured.

For reference, the size Fₚ of the external force required to press the 2-1 protruding element 121 by the length difference between the 2-1 protruding element 121 and the 2-2 protruding element 122 may be expressed as Equation 5 below.

$${F}_{p} = C {E}_{p} \frac{{L}_{p}}{{H}_{p} tan ϑ} {d}_{2}^{2} \qquad [ Equation 5 ]$$

In Equation 5, Fₚ denotes the size of the external force required to press the 2-1 protruding element 121 by the length difference between the 2-1 protruding element 121 and the 2-2 protruding element 122, C denotes the general constant, Ep denotes an elasticity coefficient of the 2-1 protruding element 121, Hp denotes a length of the 2-1 protruding element 121, Lₚ denotes a length of the base of the 2-1 protruding element 121, θ denotes an inclined angle of the 2-1 protruding element 121, and d₂ denotes the length difference between the 2-1 protruding element 121 and the 2-2 protruding element 122.

Moreover, when the stiffness k of the object 20 to be measured is uneven, the stiffness k of the object 20 to be measured in contact with the first elastic member 110 may be further derived based on a time difference between a time point t₄ when the 1-2 protruding element 112 begins to be compressed and a time point t₁ when the 1-1 protruding element 111 begins to be compressed.

Moreover, Equations 1 to 5 may assume that the object 20 to be measured and the contact portion 100 are linearly elastic, but this is only an example, and the stiffness k of the object 20 to be measured may be derived in the same or similar manner to the above-described process even when the object 20 to be measured and the contact portion 100 are hyper-elastic or viscous-elastic. Moreover, when the object 20 to be measured and the contact portion 100 are hyper-elastic or viscous-elastic, the stiffness k of the object 20 to be measured may not be linearly proportional to the ratio of the second time t₁₂ to the third time t₂₃ but may be nonlinearly proportional.

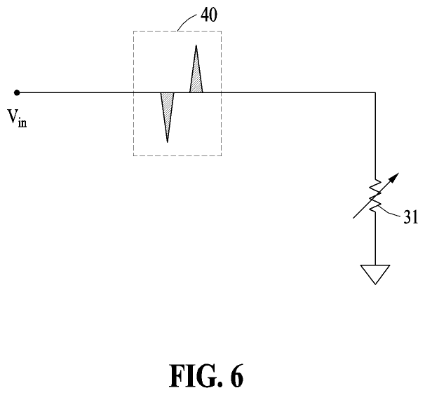

FIG. 6 schematically illustrates a circuit diagram connecting the first electrode member 31 to a first switching element 40, according to an embodiment.

Referring to FIG. 6, the stiffness measurement sensor 1 according to an embodiment may further include the first switching element 40 connected to the first electrode member 31. Likewise, the stiffness measurement sensor 1 may further include second to fourth switching elements (not shown) connected to the second to fourth electrode members 32 to 34.

The first switching element 40 and the second to fourth switching elements may switch on when the size of resistance of each of the first to fourth electrode members 31 to 34 decreases to be less than or equal to predetermined level. Moreover, the first switching element 40 and the second to fourth switching elements may switch off when the size of resistance of each of the first to fourth electrode members 31 to 34 increases to be greater than or equal to a predetermined level.

When upper parts (i.e., a part including the 1-1 protruding element 111, the 1-2 protruding element 112, the 2-1 protruding element 121, and the 2-2 protruding element 122) of the contact portion 100 and the electrode portion 30 are collectively referred to as piezoresistive elements, in general, since a signal (resistance, voltage, or current) of the piezoresistive elements continuously changes, it may be difficult to easily derive a time point when the resistance rapidly decreases.

To solve the above-described problem, when the size of resistance of the piezoresistive elements decreases to be less than or equal to a predetermined level, the first switching element 40 and the second to fourth switching elements may switch on so that a time point when the 1-1 protruding element 111, the 1-2 protruding element 112, the 2-1 protruding element 121, and the 2-2 protruding element 122 are compressed or a time point when the size of resistance of the piezoresistive elements decreases to be less than or equal to a predetermined level may be easily derived. That is, a type of signal-to-noise ratio may increase so that the time point when the size of resistance decreases to be less than or equal to a predetermined level may be easily derived.

Moreover, the first switching element 40 and the second to fourth switching elements are described herein as simply switching on or off at a certain resistance value or certain applied voltage, but this is only an example, and it is obvious that the first switching element 40 and the second to fourth switching elements may be elements that generate a spike signal when switched on as an ovonic threshold switching (OTS) switching element if necessary, and the spike signal disappears when switched off. Moreover, when the OTS switching element is used, the time point when the size of resistance decreases to be less than or equal to a predetermined level may be easily derived based on a spike generation time point. When a frequency of the spike is 1 megahertz (MHz), a spike interval is 1 microsecond (μs), so an error at the start time point when the spike is derived may be extremely low to the level of 1 μs.

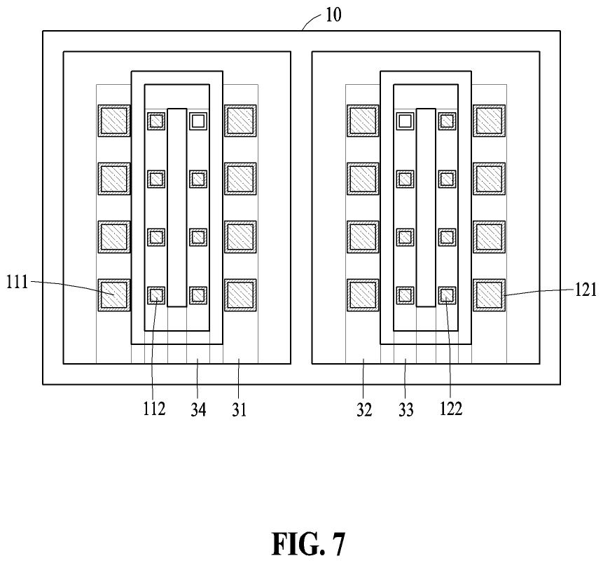

FIG. 7 is a cross-sectional view taken along line A-A of FIG. 1 to show a circuit diagram in which the first to fourth electrode members 31 to 34 are electrically connected to each other, according to an embodiment.

According to an embodiment, each of the 1-1 protruding element 111, the 1-2 protruding element 112, the 2-1 protruding element 121, and the 2-2 protruding element 122 may include a plurality of protruding elements, and each of the first to fourth electrode members 31 to 34 may form a closed circuit.

Here, according to the number of each of the 1-1 protruding element 111, the 1-2 protruding element 112, the 2-1 protruding element 121, and the 2-2 protruding element 122, and a conductivity value of a conductive material coated on a upper end of each of the 1-1 protruding element 111, the 1-2 protruding element 112, the 2-1 protruding element 121, and the 2-2 protruding element 122, the reactivity and sensitivity of each of the piezoresistive elements may be controlled.

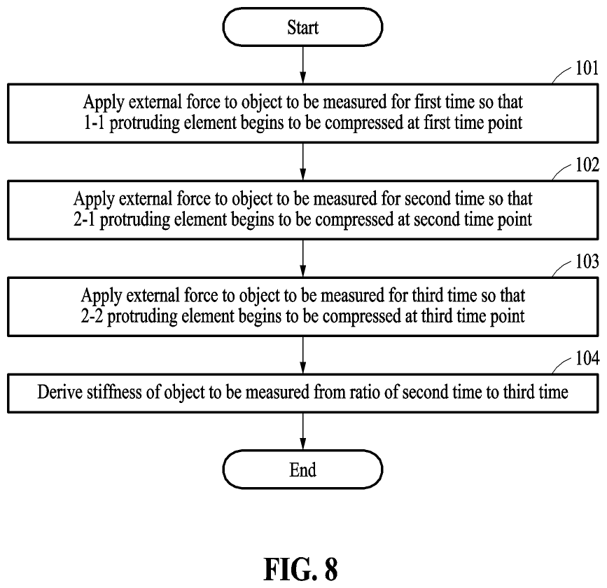

FIG. 8 is a flowchart illustrating a method of driving the stiffness measurement sensor 1, according to an embodiment.

Referring to FIGS. 2 to 4 and FIG. 8, a method of driving the stiffness measurement sensor 1 according to an embodiment is described below.

First, in operation 101, an external force may be applied to the object 20 to be measured for a first time so that the 1-1 protruding element 111 may begin to be compressed at the first time point t₁, and thus the contact resistance of the 1-1 protruding element 111 with the first electrode member 31 may rapidly decrease.

Next, in operation 102, the external force may be applied to the object 20 to be measured for the second time t₁₂ so that the 2-1 protruding element 121 may begin to be compressed at the second time point t₂, and thus the contact resistance of the 2-1 protruding element 121 with the second electrode member 32 may rapidly decrease.

Next, in operation 103, the external force may be applied to the object 20 to be measured for the third time t₂₃ so that the 2-2 protruding element 122 may begin to be compressed at the third time point t₃, and thus the contact resistance of the 2-2 protruding element 122 with the third electrode member 33 may rapidly decrease.

From operations 102 and 103, in operation 104, the ratio of the second time t₁₂ to the third time t₂₃ may be obtained, and from this, the stiffness k of the object 20 to be measured may be derived according to Equations 1 to 4 described above.

As described above, the stiffness measurement sensor 1 according to an embodiment may derive the stiffness k of the object 20 to be measured without being affected by the speed v at which the external force is applied to the object 20 to be measured.

In addition, the stiffness measurement sensor 1 according to an embodiment may be manufactured in a small size, so there may be no problem with durability of the device due to detachment and/or attachment.

While the embodiments are described with reference to drawings, it will be apparent to one of ordinary skill in the art that various alterations and modifications in form and details may be made in these embodiments without departing from the spirit and scope of the claims and their equivalents. For example, suitable results may be achieved if the described techniques are performed in a different order, and/or if components in a described system, architecture, device, or circuit are combined in a different manner, and/or replaced or supplemented by other components or their equivalents.

Accordingly, other implementations are within the scope of the following claims.

1. A multi-level mechanoreceptor device-based stiffness measurement sensor comprising:
a substrate;
a contact portion disposed below the substrate and partially spaced apart from the substrate; and
an electrode portion disposed at a lower end of the substrate and disposed corresponding to the contact portion,
wherein an area where the electrode portion and the contact portion are in contact gradually increases as an external force is applied to an object to be measured at a bottom part of the contact portion.

2. The multi-level mechanoreceptor device-based stiffness measurement sensor of claim 1, wherein the contact portion comprises a first elastic member and a second elastic member capable of being pressed by the external force,
wherein the first elastic member is thicker than the second elastic member,
wherein a lower end of the first elastic member contacts an upper surface of the object to be measured, and
wherein a lower end of the second elastic member is spaced apart from the upper surface of the object to be measured.

3. The multi-level mechanoreceptor device-based stiffness measurement sensor of claim 2, wherein
the first elastic member comprises a 1-1 protruding element positioned at an upper end of the first elastic member,
the second elastic member comprises a 2-1 protruding element and a 2-2 protruding element positioned at an upper end of the second elastic member, and the 2-1 protruding element has a protruding length that is longer than that of the 2-2 protruding element, and
the electrode portion comprises a first electrode member, a second electrode member, and a third electrode member disposed at a position corresponding to the 1-1 protruding element, the 2-1 protruding element, and the 2-2 protruding element, respectively.

4. The multi-level mechanoreceptor device-based stiffness measurement sensor of claim 3, wherein
when the external force is applied to the object to be measured for a first time, the object to be measured contracts and the first elastic member contracts so that the 1-1 protruding element begins to contract at a first time point, and thus a contact resistance of the 1-1 protruding element with the first electrode member rapidly decreases,
when the external force is applied to the object to be measured for a second time, the object to be measured contracts, and the first elastic member and the second elastic member contract so that the 2-1 protruding element begins to contract at a second time point, and thus a contact resistance of the 2-1 protruding element with the second electrode member rapidly decreases, and
when the external force is applied to the object to be measured for a third time, the object to be measured contracts, and the first elastic member and the second elastic member contract so that the 2-2 protruding element begins to contract at a third time point, and thus a contact resistance of the 2-2 protruding element with the third electrode member rapidly decreases, and
wherein the second time is defined as a time from the first time point to the second time point, and the third time is defined as a time from the second time point to the third time point.

5. The multi-level mechanoreceptor device-based stiffness measurement sensor of claim 4, wherein
the second time is inversely proportional to a speed at which the external force is applied to the contact portion, and
the third time is inversely proportional to a speed at which the external force is applied to the contact portion and stiffness of the object to be measured.

6. The multi-level mechanoreceptor device-based stiffness measurement sensor of claim 5, wherein a ratio of the second time to the third time is proportional to the stiffness of the object to be measured.

7. The multi-level mechanoreceptor device-based stiffness measurement sensor of claim 3, further comprising:
a first switching element, a second switching element, and a third switching element connected to the first electrode member, the second electrode member, and the third electrode member, respectively,
wherein each of the first switching element, the second switching element, and the third switching element switches on when a size of a contact resistance decreases to be less than or equal to a predetermined level.

8. The multi-level mechanoreceptor device-based stiffness measurement sensor of claim 3, wherein
each of the 1-1 protruding element, the 2-1 protruding element, and the 2-2 protruding element comprises a plurality of protruding elements, and
each of the first electrode member, the second electrode member, and the third electrode member forms a closed circuit.

9. A method of driving a multi-level mechanoreceptor device-based stiffness measurement sensor, the method comprising:
applying an external force to an object to be measured for a first time so that a 1-1 protruding element begins to be compressed at a first time point, and thus a contact resistance of the 1-1 protruding element with a first electrode member rapidly decreases;
applying the external force to the object to be measured for a second time so that a 2-1 protruding element begins to be compressed at a second time point, and thus a contact resistance of the 2-1 protruding element with a second electrode member rapidly decreases;
applying the external force to the object to be measured for a third time so that a 2-2 protruding element begins to be compressed at a third time point, and thus a contact resistance of the 2-2 protruding element with a third electrode member rapidly decreases; and
deriving stiffness of the object to be measured from a ratio of the second time to the third time.
